(19) United States
(12) Patent Application Publication
(10) Pub. No.: US 2009/0252926 A1
(43) Pub. Date: Oct. 8, 2009

(54) THIN-WALLED CALENDERED PTFE

(75) Inventors: Jamie S. Henderson, Oakland, NJ (US); Matt Kebabjian, Whippany, NJ (US); Calin Druma, Cupertino, CA (US); Matthew Noesner, West Orange, NJ (US)

Correspondence Address: VIDAS, ARRETT & STEINKRAUS, P.A., SUITE 400, 6640 SHADY OAK ROAD, EDEN PRAIRIE, MN, 55344, US

(73) Assignee: BOSTON SCIENTIFIC SCIMED, INC., Maple Grove, MN (US)

(21) Appl. No.: 12/061,898

(22) Filed: Apr. 3, 2008

(51) Int. Cl. A61F 2/06; B32B 27/00; A61F 2/00; B29C 43/24

(52) U.S. Cl. 428/156; 264/175; 623/11.11; 428/219; 623/1.15

(57) ABSTRACT

The invention relates to prosthetic implants, and particularly those prosthetic implants which have an improved water entry pressure resistance. Particularly, the invention relates to a strong calendered PTFE tube and processes for making a strong calendered PTFE tube. The calendered PTFE tube may then be used to form a strong calendered expanded PTFE tube.

### FIELD OF THE INVENTION

The invention relates to prosthetic implants. Particularly, this invention relates to a strong calendered PTFE tube and processes for making a strong calendered PTFE tube. The calendered PTFE tube may then be used to form a strong calendered expanded PTFE tube.

### BACKGROUND OF THE INVENTION

It is well known to use extruded tubes of polytetrafluoroethylene (PTFE) as implantable intraluminal prostheses, particularly as vascular grafts. PTFE is particularly suitable as an implantable prosthesis as it exhibits superior biocompatibility. If desired, PTFE may be made into a tube, which may be used as a replacement or to repair blood vessels or other bodily vessels. PTFE, particularly expanded PTFE, exhibits superior biocompatibility and low thrombogenicity, which makes it particularly useful as vascular graft material in the repair or replacement of blood vessels or other bodily lumens.

Such vascular grafts may be used to replace, reinforce, or bypass a diseased or injured body lumen. One conventional method of manufacturing “expanded” PTFE layers is described in U.S. Pat. No. 3,953,566 by Gore. In the methods described therein, a PTFE paste is formed by combining a PTFE resin and a lubricant. The PTFE paste may be extruded. After extrusion, the PTFE article is stretched to create a porous, high strength PTFE article. The expanded PTFE (ePTFE) layer is characterized by a porous, open microstructure that has nodes interconnected by fibrils.

Structures formed of ePTFE exhibit certain beneficial properties as compared with textile prostheses. The expanded PTFE structure has a unique configuration defined by nodes interconnected by fibrils. The space between the nodes that is spanned by the fibrils is defined as the internodal distance (IND). Such an expansion process increases the volume of the PTFE layer by increasing the porosity, which results in a decrease in the density and increase in the internodal distance between adjacent nodes in the microstructure. The node and fibril structure defines pores in the structure that facilitate a desired degree of tissue ingrowth while remaining substantially fluid-tight.

Typically, porous expanded PTFE is desirable to promote tissue growth; however, its porosity may render it susceptible to leakage of fluids through the wall. Typical ePTFE structures may be susceptible to water entry pressures of less than 10 psi. The level of water entry pressure is commonly known as the WEP value. A lower WEP value means that the level of pressure required for water to leak through the structure is lower, making the structure less desirable. Structures with a lower WEP value may in turn create a phenomenon known as ultra-filtration which can ultimately lead to an increase in the abdominal aortic aneurysm diameter. As such, there is a need in the art for a strong ePTFE tube with very low or zero permeability and a higher water entry pressure level.

Calendering is a process well known in the production of PTFE tape and film material that adds significant strength to the finished product. Generally, calendering involves feeding an extruded PTFE tape between two hot rollers and applying enough tension on the take-up to eliminate the material from welding. The calender rollers compress the tape in the machine direction and squeeze it to a small fraction of the original thickness. Calendering in the transverse direction particularly increases the strength of the PTFE base material. This highly fibrillated tape is many times stronger than the original extrudate and still may be expanded. Fibrillation is the process by which PTFE polymer strands are moved via mechanical means from the base polymer sphere, otherwise known as raw dispersion particle (“RDP”). After calendering, the material can then be expanded to form ePTFE.

Current methods, including those using two hot rollers as described above, are useful for calendering sheets or tapes of PTFE extrusions. However, these processes may be less desirable for calendering tubes of PTFE, as they would likely collapse the lumen of the tube, rendering it useless as a tubular implant. Accordingly, there is a desire in the art for a method of calendering a tube of a PTFE, which contains a lumen. Further, there is a need for a modified PTFE structure which can be expanded after calendering.

### SUMMARY OF THE INVENTION

In one aspect of the invention, there is provided a prosthesis including: a calendered tubular body including ePTFE having a densified node and fibril structure, the tubular body having a water entry pressure value of at least 20 psi, a thickness of between 50 to 100 microns, and a density of between about 0.5 g/cc to about 1.0 g/cc.

In another aspect of the invention, there is provided a method for forming a PTFE tube including the steps of: forming a green PTFE tube with an inner and outer surface; positioning the green PTFE tube on a tubular support surface having an outer surface, wherein the inner surface of the green PTFE tube is associated with the outer surface of the tubular support surface; and applying a compressive force along the length of the outer surface of the green PTFE tube. The compressed PTFE tube may then optionally be expanded to form ePTFE.

### DESCRIPTION OF THE DRAWINGS

FIG. 1 shows a cross-sectional representation of one embodiment of the calendering mechanism of the present invention.

FIG. 2 shows a side-angled view of one embodiment of the calendering mechanism of the present invention.

FIG. 3 shows an alternate embodiment of the calendering mechanism of the present invention.

FIG. 4 shows an alternate embodiment of the calendering mechanism of the present invention.

FIG. 5 shows an alternate embodiment of the calendering mechanism of the present invention incorporating a rolling rod.

### DETAILED DESCRIPTION OF THE INVENTION

Typically, PTFE is formed via extruding a blend of PTFE resin through an extruder die. PTFE may be extruded in any number of different forms, including tubular, sheet-like, rounded, or other shape desired. The extruded PTFE may be treated in any number of ways; in particular it may be calendered to increase the strength of the wall of the tubing. Such calendering may take place at any time after the PTFE has been extruded. In one embodiment, the calendering may take place immediately after extrusion. Preferably, the calendering is performed while the PTFE is in a fully wetted state. In such embodiments, the calendered PTFE may then subsequently be expanded to form expanded PTFE (ePTFE). If desired, a calendering tool or tools may be associated with the extruder die exit to calender the extrudate as it is being extruded. Calendering the PTFE helps increase the density of the material, imparting increased strength and stability to the material, and providing increased strength and stability to expanded PTFE. It is desirable to calender a tube of PTFE without collapsing the lumen of the tube.

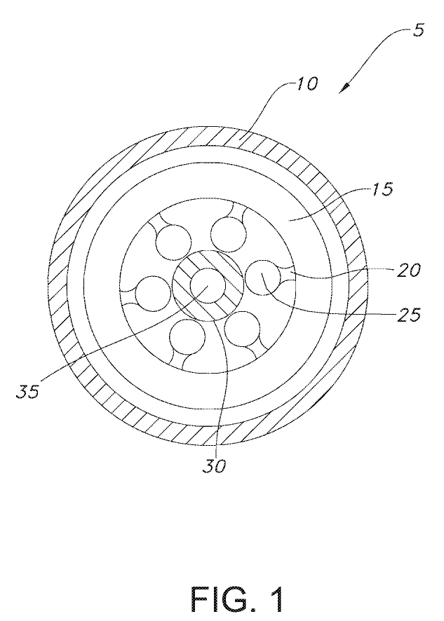

In one embodiment incorporating a calendering tool associated with the extruder die, an iris of highly polished rollers may be included. With reference to FIG. 1, an iris 5 is shown. In this embodiment, the iris 5 includes a stationary ring 10 forming the outside of the iris 5. Inside the stationary ring 10 is a rotating iris ring 15, which may spin in a clockwise or counterclockwise rotation, or it may spin in a varying rotation. Associated with the inside layer of the rotating iris ring 15 are a plurality of roller standoffs 20. These roller standoffs 20 are adjustable in height, depending on the thickness of the PTFE desired. There may be any number of roller standoffs 20 desired. In a preferred embodiment, there are six roller standoffs 20, although there may be three to twelve roller standoffs 20.

On the ends of each of the roller standoffs 20 is at least one roller 25. The roller 25 is preferably spherical in shape and is preferably highly polished to provide a smooth calendered effect. The rollers 25 are preferably positioned such that they may rotate in any number of directions, preferably rotating in the same direction as the rotation of the rotating iris ring 15.

The PTFE tube 30 is fed through the iris 5. The PTFE tube 30 has an inner surface and an outer surface. As shown in FIG. 1, the rollers 25 contact the PTFE tube 30 on its outer surface. The PTFE tube 30 is preferably disposed around a support surface, such as mandrel tip 35, which helps form and support the inner surface of the PTFE tube 30. The inner surface of PTFE tube 30 is preferably in contact with the outer surface of mandrel tip 35. Preferably the mandrel tip 35 is highly polished and smooth, giving a sheer smooth effect on the inside surface of the PTFE tube 30. If desired, the rollers 25 and/or the mandrel tip 35 may be grooved or include protrusions to give a textured tube after calendering.

To calender the tube 30 after extrusion, the PTFE tube 30 enters the middle portion of iris 5, where it may be calendered to any thickness desired. The iris 5 may be designed to mechanically constrict upon the outer surface of the PTFE tube 30, such that the rollers 25 are in substantial contact with the PTFE tube 30. The constriction of the iris 5 exerts mechanical compression on the outer surface of the PTFE tube 30. Preferably, each roller 25 exerts substantially the same level of pressure on PTFE tube 30. As the rotating iris ring 15 and the rollers 25 rotate, the PTFE tube 30 travels axially through the iris 5. The PTFE tube 30 is thereby compressed onto the mandrel tip 35, and exits the iris 5 more compressed than prior to its entry. Depending on the calendering speed, roller positioning, level of pressure exerted and temperature of the rollers, the tube may be calendered down to produce a tube of any suitable thickness. The reduction ratio of an embodiment of the calendering process, which is a ratio of the thickness of the original tube 30 to the thickness of the calendered tube, may be between about 3:1 to about 75:1, and most preferably is between about 7.5:1 to about 15:1. The compression is desirably capable of compressing the original tube 30 to as low as 1% of its original thickness. The tube 30 may travel through the iris 5 for any desired length of time, and is most preferably disposed within the iris 5 for about 30 seconds to about one minute. However, depending upon the degree of compression desired, the tube 30 may be within the iris 5 for as little as 10 seconds and as much as 5 minutes.

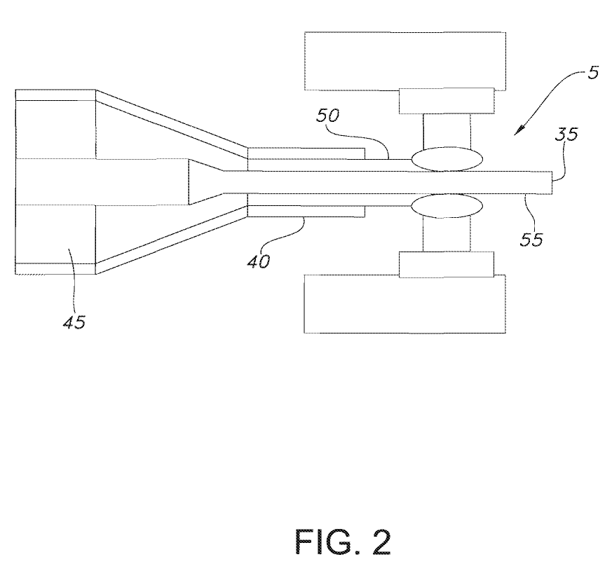

As seen in FIG. 2, the iris 5 may be associated with an extruder nozzle 40, being located at a point which is proximate to the exit point of the PTFE 45. In one embodiment, the unextruded PTFE 45 travels through the extruder nozzle 40, forming an extrudate of non-compressed PTFE tubing 50. Shortly after the extruded (non-compressed) PTFE tube 50 is extruded from the extruder nozzle 40, it may enter the iris 5, where the uncompressed PTFE tube 50 may form a calendered tube 55 of a desired thickness. As demonstrated in FIG. 2, the non-compressed PTFE tube 50 is substantially thicker than the post-calendered PTFE tube 55.

In some embodiments, a mandrel 35 may extend through and beyond the extrusion nozzle 40 to support and shape the lumen of the extruded tube 50 as it is being calendered. In a preferred embodiment, the mandrel 35 protrudes out of the front of the extruder nozzle 40, extending through the center of the iris 5. In this embodiment, the uncompressed PTFE tube 50 exits the extruder nozzle 40, traveling along the mandrel 35, which gives support to the uncompressed tube 50 as it travels through the iris 5. The iris may be positioned anywhere desired, including at a location directly after extrusion, or any location. Alternatively, the iris 5 may be disassociated from the extrusion nozzle 40. In such embodiments, the PTFE tube 50 is first extruded and formed. At a later point in time, when desired, the uncompressed tube 50 may be calendered via the processes described above.

In some embodiments, a take-up system may be included. The take-up system may be used to prevent the diameter of the calendered tube 55 from increasing as it is calendered through the iris 5. In some embodiments, the take-up system may be used to intentionally increase the diameter of the calendered tube 55.

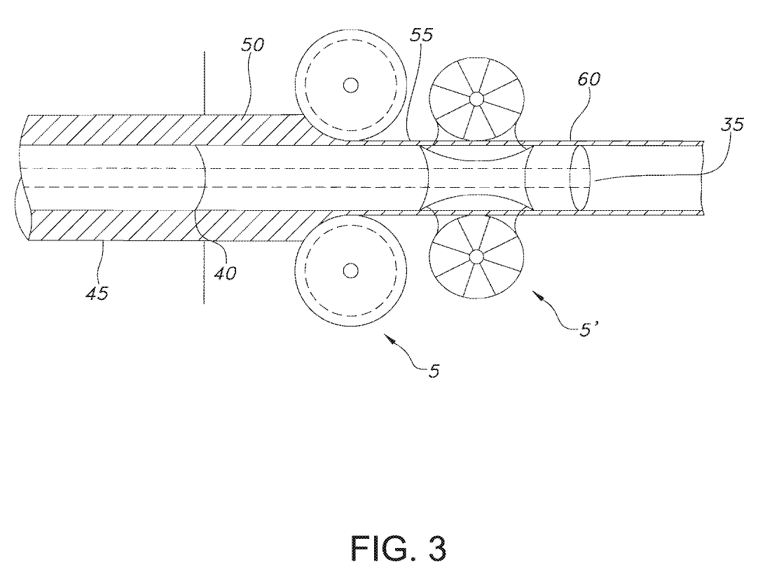

In one embodiment, the iris 5 may include multi-sectional components, including counter-rotating sections and other rotating elements to provide various twists to the compressed tube 55. The sections may be used to counteract any twisting which may have occurred in the calendered tube 55. In some embodiments, the calendering mechanism may include a plurality of irises 5. In this embodiment, the irises 5 may be located sequentially as the PTFE tube 50 travels axially along the support mandrel 35. The multiple irises 5 may be preset to increase the amount of squeezing and/or calendering with each sequential iris 5 as the PTFE tube 50 enters each sequential iris 5. FIG. 3 shows an embodiment using multiple irises 5, 5′ axially located along the path that the PTFE tube 50 travels. PTFE resin 45 exits the extruder nozzle 40, and is indexed through the first iris 5, where it forms a first calendered tube 55. The first calendered PTFE tube 55 is then indexed through a second sequential iris 5′, where it forms a second calendered tube 60. The second calendered PTFE tube 60 then exits the system. As explained above, any number of irises 5 may be incorporated into the system.

In some embodiments, the calendering mechanism may be set to calender only portions of the uncompressed tube 50 using rollers 25. In some embodiments, the portioned calendering may create undisturbed diamond shaped sections of tube 55 between each set of irises 5. The calendering mechanism may also be set to overlay cross helical patterns through the plurality of irises 5 as the extruded tube 50 continues through the calendering mechanism. A plurality of cross helical patterns may be created on the calendered tube 55, thus establishing an offset calendering configuration throughout the entire length of the calendered tube 55. Accordingly, the entire surface of the calendered tube 55 may be effectively calendered with this technique.

The rollers 25 may be selected from any shape of calendering heads desired, including cylindrical rolls, 3D tapered helical rolls, spheres, rotating disks, driven rollers, or combinations thereof. Further, the rollers 25 may be profiled with axes at an angle with respect to the extrusion direction. It is preferred that there are a sufficient number of rollers 25 incorporated in the iris 5 so as to calender the tube 50 a full 360 degrees circumference. If desired, there may be multiple sets of rollers 25 surrounding the outside of the uncompressed tube 50 to effectively calender the tube 50 circumferentially. The size and shape of the rollers 25 may be any size and shape desired, such that the outer layer of the tube 50 is in substantial contact with the rollers 25 as it is indexed through the iris 5. The rollers 25 may push, pull and/or twist the tube 50 as it is extruded, depending on the choice of calendering head and the effect desired.

The amount of thinning of the tube 55 may depend on several variables, including the pressure applied on the outside of the uncompressed tube 50, the amount of time the rollers 25 are in contact with the tube 50, the relative temperature of the rollers 25 and mandrel 35, the amount of lubricant applied to the components of the iris 5 during calendering of the tube 50, and the type of lubricant utilized in the process of strengthening the calendered tube 55.

The calendering temperatures and processing parameters may be chosen so that the tube is calendered sufficiently. The rollers may be any desired temperature, and are preferably at an elevated temperature not exceeding about 327° C. Preferably, the rollers 25 may be heated to a temperature between about 100° F. and about 300° F., and specifically between about 120° F. and about 160° F. during the calendering process. Depending upon the level of pressure exerted, the rollers may be lower in temperature, as low as room temperature.

In some embodiments, the calendered tube 55 may be stretched after calendering. In these instances, the tube 55 may be calendered to a thickness that is only slightly greater than a final desired thickness, so that the final stretch of the tube 55 causes the expanded tube to have its desired thickness. Once the tube 50 has been calendered to the desired thickness, the calendered tube 55 may optionally be mechanically stretched transversely (also called the cross machine direction), in the longitudinal direction (also called the machine direction), both of these directions or any other suitable direction or combination of directions, in order to thin the calendered tube 55, generate a suitable microstructure and mechanically work the material.

Typically, the calendered tube 55 formed via the processes discussed herein may have a density ranging from about 0.4 to about 2 grams/cc; specifically, from about 1.0 to about 1.5 grams/cc. In embodiments where the PTFE is calendered while it is still in a wetted state, the density of the resulting tube shows increased density. Density of products which are calendered when the PTFE tube is still in a wetted state is preferably about 1.8 to about 2.0 g/cc. The nodal spacing of an expanded PTFE tube after calendering may range from about 0.5 to about 200 microns; specifically, from about 5 to about 35 microns. Preferably, the expanded PTFE tube formed by the processes described herein has a WEP value of at least 15 psi, and more preferably at least 20 psi.

The calendering force may include both an axial force as well as a tangential force on the PTFE tube 50. The tangential calendering force may promote radial movement of the partially fibrillated RDPs, promoting cross-fibrillation of the extruded tube 50 and further enhancing the radial strength of the calendered tube 55. The friction force between the rollers 25 and the tube 50 may be enhanced by applying a polyurethane coating to the rollers 25 and/or the mandrel 35. Either or both of the rollers 25 and the mandrel 35 may be heated during calendering of the tube 50. Heating has a tendency to further compress and thin the calendered tube 55. Any degree of pressure and friction force may be exerted. The greater the pressure exerted on the tube 50, the thinner the resulting calendered tube 55. Preferably, the rollers 25 exert a pressure on the tube 50 of about 100 psi to about 700 psi and most preferably of from about 300 psi to about 500 psi. The pressure exerted may be reduced if a thicker calendered tube 55 is desired, or if the rollers 25 and/or the mandrel 35 are heated.

The iris 5 may optionally have driving rollers or shaping cam followers, which rotate inline with the direction that the tube 50 is traveling through the iris 5. The driving rollers may include a curved outer surface, preferably having a radius approximately that of the desired calendered tube 55.

The calendered tube may be expanded to form ePTFE if desired. Expansion of PTFE creates a node-fibril structure, and further imparts significant strength to the expanded tube. The rate at which expansion is performed may play a role in the strength of the fibers and of the resultant structure. Early calendering of the PTFE structure may additionally play a role in the strength of the resultant expanded structure. Expansion may be of any desired ratio of expanded structure to unexpanded structure, including from about 15:1 to about 100:1. Further the rate of expansion may be any rate desired, and generally ranges from up to about 400%/second to about 1000%/second. Expansion may take place at rates higher than 1000%/second if desired. Further, expansion may take place at rates lower than about 400%/second if desired. Expansion of the calendered PTFE structure additionally imparts greater flexibility to the structure, with the level of flexibility depending upon the rates and ratios of expansion.

The process described herein may optionally incorporate the use of a lubricant applied to the components of the iris 5, either on the various components, in the PTFE resin 45, or on the tube 50 itself. Lubrication aids in smoothly indexing the tube 50 through the system, and may help prevent bunching and potential cracking or breaking of the tube 50 as it is calendered. Any type of lubricant may be used during calendering of the tube 50. Preferably, the components or the tube 50 have a coating or layer of isopar K and/or isopar M. In addition to any lubricant applied to the outer surfaces of the iris 5, the amount and type of lubricant used in the PTFE resin 45 during extrusion of the tube 50 will additionally play a role in determining the consistency of the calendered tube 55. Preferably, the lubricant used is isopar K or M, and the content is preferably about 18% or greater. The PTFE resin 45 may be any type desired, and preferably is a homopolymer or modified homopolymer of PTFE. It is preferred that the tubing 50 remain sufficiently lubricated throughout the calendering process.

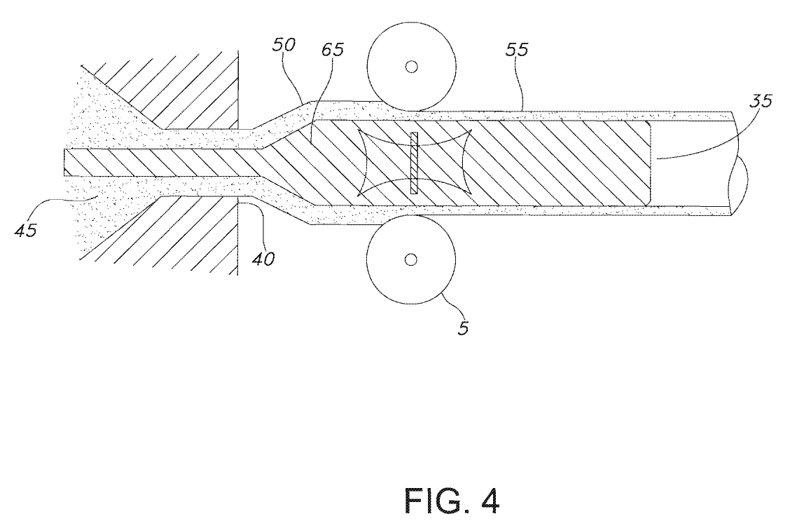

The tubing 50 may be enlarged in diameter at any point of the system if desired. In one particular embodiment, the tubing 50 may be enlarged prior to calendering, such as via a bulbous extrusion tip 65 at or about the location of the extruder nozzle 40, such as shown in FIG. 4. As seen in FIG. 4, PTFE resin 45 may be extruded through the extruder nozzle 40 to form an extruded, uncalendered PTFE tube 50. After extrusion, the PTFE tube 50 may be enlarged as it is indexed over a bulbous extrusion tip 65. After enlargement, the enlarged PTFE tube 50 enters an iris 5 where it is calendered, resulting in a calendered PTFE tube 55. As described above, the iris 5 of this embodiment may incorporate several rollers 25 to effect calendering circumferentially. In another embodiment, the tubing 50 may be first calendered, and then subsequently enlarged. Further, the system may use several sequential irises 5, as previously discussed.

In another embodiment, the rollers 25 of the iris 5 may be substituted with pins. In this embodiment, the pins may be driven, while the housing of the pins remains stationary. The rolling pins may be disposed at various points circumferentially about the outer surface of the tube 50 as it is indexed through the iris 5.

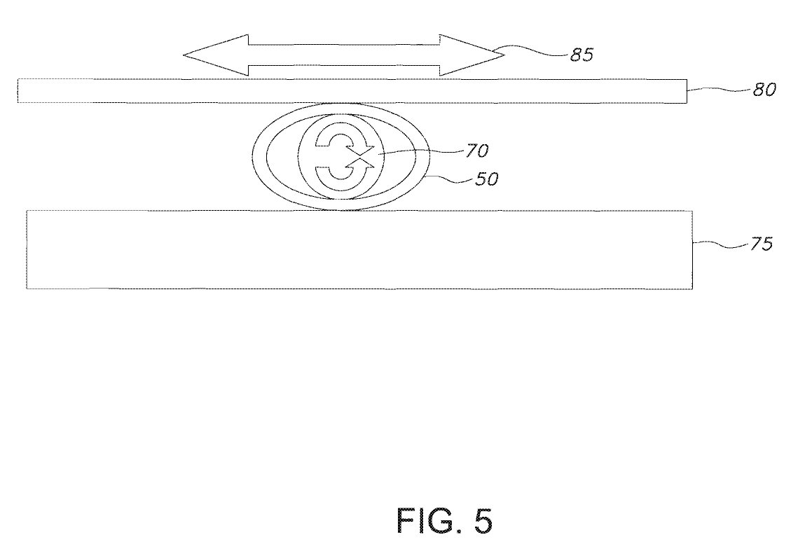

As depicted in FIG. 5, in an alternate embodiment, the PTFE tube 50 may be calendered at a time after it has been extruded via using a rod 70 that is not associated with the extrusion nozzle 40. In this embodiment, the PTFE green tube 50 is extruded and collected. After the tube 50 has been collected and removed from the extruder 40, a rounded rod 70 may be inserted into the lumen of the uncalendered tube 50. Preferably, the diameter of the rod 70 is less then inside diameter of the PTFE tube 50. Optionally, the tube 50 may be soaked with a lubricant, such as isopar G, prior to the insertion of the rod 70.

Once the rod 70 is inserted into the lumen of the tube 50, the rod 70 and tube 50 may be rolled on a flat surface 75, or optionally between a bottom flat surface 75 and top flat surface 80. The rod 70 is preferably maintained within the lumen of the tube 50 during the rolling process. Any rolling techniques may be used depending on the desired calendering effect. Additionally, any degree of pressure may be used while rolling, where a higher pressure exerted on the tube 50 will result in a thinner calendered tube 55. Optionally, the system may be heated, either by using heated lubricant, or by heating the elements of the rolling system, including the rod 70, the bottom flat surface 75 or the top flat surface 80.

The rod 70 may be inserted in specific locations in the lumen of the tube 50, may be inserted only partially within the lumen of the tube 50, or it may be inserted fully through the entire length of the tube 50. Preferably, the tube 50 is rolled using a back and forth rolling motion, continuing the rolling motion until the desired thickness is achieved. This method results in a calendered tube, which is calendered in the transverse direction, adding substantial strength and stability to the calendered tube.

In any process described herein, the tube 50 may be expanded and/or sintered prior to the calendering methods described herein. In other embodiments, the calendered tube 55 may be expanded and/or sintered after the calendering process has completed.

1. A prosthesis comprising:
a calendered tubular body comprising ePTFE having a densified node and fibril structure, said tubular body having a water entry pressure value of at least 20 psi, a thickness of between 50 to 100 microns, and a density of between about 0.5 g/cc to about 1.0 g/cc.

2. The prosthesis of claim 1, wherein said tubular body comprises an inner surface and an outer surface.

3. The prosthesis of claim 1, wherein said outer surface of said tubular body comprises a plurality of helical patterns.

4. The prosthesis of claim 1, wherein said outer surface of said tubular body comprises a plurality of cross-helical patterns.

5. A method for forming a PTFE tube comprising the steps of:
forming a green PTFE tube with an inner and outer surface;
positioning said green PTFE tube on a tubular support surface having an outer surface, wherein said inner surface of said green PTFE tube is associated with said outer surface of said tubular support surface; and
applying a compressive force along the length of said outer surface of said green PTFE tube.

6. The method of claim 5, wherein said compressive force is applied by a calendering mechanism comprising at least one rotatable roller, positioned about the outer surface of said green PTFE tube.

7. The method of claim 6, wherein said at least one rotatable roller is selected from the group consisting of cylindrical rollers, 3-dimensional tapered helical profiled rollers, and driven rollers.

8. The method of claim 6, wherein said at least one rotatable roller is advanced along at least a portion of the length of said green PTFE tube.

9. The method of claim 6, wherein said green PTFE tube is advanced along its longitudinal axis as it passes through said calendering mechanism.

10. The method of claim 6, wherein said calendering mechanism includes an iris attachment for positioning said at least one rotatable roller radially about said outer surface of said green PTFE tube.

11. The method of claim 6, wherein said green PTFE tube is advanced along its longitudinal axis through said iris attachment as said at least one rotatable roller rotates.

12. The method of claim 6, further comprising the step of heating said calendering mechanism.

13. The method of claim 5, wherein said application of compressive force is applied by shaping cam followers which rotate inline with the movement of the green tube along the tubular surface.

14. The method of claim 6, further comprising the step of creating a multitude of helical or cross-helical patterns on said outer surface of said green PTFE tube.

15. The method of claim 5, wherein said step of applying compressive force comprises rolling said tubular support surface against at least one external surface, wherein said at least one external surface contacts said outer surface of said green PTFE tube.

16. The method of claim 17, comprising two external surfaces, wherein said rolling tubular support surface is located between said external surfaces.

17. The method of claim 5, further comprising the step of expanding said PTFE tube after said step of applying a compressive force along the length of said outer surface of said green PTFE tube.
